(19) United States
(12) Patent Application Publication
(10) Pub. No.: US 2025/0032298 A1
(43) Pub. Date: Jan. 30, 2025

(54) SPINAL DECOMPRESSION DEVICE WITH ACTIVE LORDOTIC SUPPORT

(71) Applicant: Excite Medical of Tampa Bay, LLC, Tampa, FL (US)

(72) Inventors: Scot Leslie Johnson, Tampa, FL (US); Saleem Najeeb Musallam, Tampa, FL (US)

(21) Appl. No.: 18/231,620

(22) Filed: Aug. 8, 2023

Related U.S. Application Data
(60) Provisional application No. 63/396,033, filed on Aug. 8, 2022.
(60) Provisional application No. 63/396,364, filed on Aug. 9, 2022.
(60) Provisional application No. 63/396,668, filed on Aug. 10, 2022.

(51) Int. Cl. A61F 5/042

(52) U.S. Cl. CPC A61F 5/042; A61F 2250/0004

(57) ABSTRACT

A spinal decompression device allowing for dynamic adjustment of the application of pressure and support to the apex of lordosis of a patient during treatment. The device incorporates an inflatable lordotic support bladder positioned and oriented to apply an adjustable and variable degree of support and pressure to the apex of lordosis of the patient's spine during spinal decompression according to a predefined treatment protocol or real-time dynamic adjustment under the control of a medical provider or patient. In certain embodiments, the device may recall settings for a patient based on prior treatment. In other embodiments, the treatment may be adjusted based on biofeedback.

### FIELD OF THE DISCLOSURE

The present disclosure is directed generally to a spinal decompression device, and in particular, to a spinal decompression device incorporating an active lordotic support mechanism.

### SUMMARY OF THE PRESENT DISCLOSURE

The present disclosure is directed to a device for spinal decompression. In particular, the present disclosure is directed to a spinal decompression device incorporating an inflatable lordotic support bladder housed within the treatment bed of the device. The inflatable lordotic support bladder disclosed herein is positioned and oriented such that its apex when inflated is located underneath an anatomical location on a patient known as an “apex of lordosis.” In certain embodiments, the inflatable lordotic support bladder is disposed within an upper mattress portion of a spinal decompression device. The inflatable lordotic support bladder is inflated as set forth herein, to accommodate a patient's specific lumbar and thoracic spine posture as presented at the specific time of treatment.

The present disclosure allows a healthcare provider to prescribe an appropriate treatment bed morphology by setting the fill pressure of the inflatable lordotic support before and during a spinal decompression treatment. This capability is referred to herein as “Lordotic Support Adjustment.” The present disclosure allows a healthcare provider to prescribe both limits on, and adjustments to, the configuration of a modern spinal decompression device prior to and during a spinal decompression treatment. In one embodiment, the device includes a handheld remote which is used by a healthcare provider to make adjustments to treatment parameters, which may include the height and/or pressure of the inflatable lordotic support housed within an upper mattress.

The present disclosure describes devices for increasing patient safety and ensuring a rate of spinal elongation which promotes relaxation of paraspinal muscles. The present disclosure also allows for modification of the shape and level of support of a critical region of the treatment bed located underneath the patient's lumbar spine, via the inflatable lordotic support.

### BRIEF DESCRIPTION OF THE DRAWING FIGURES

The foregoing disclosure will be best understood, and the advantages thereof made most clearly apparent, when consideration is given to the following detailed description in combination with the drawing figures presented. The detailed description makes reference to the following drawings:

FIG. 1 is a side view of a spinal decompression device;

FIG. 2 is a side-end view of the spinal decompression device of FIG. 1;

FIG. 3 is a side view of a bed platform of the spinal decompression device of FIGS. 1 and 2;

FIG. 4 is a top view of the bed platform of FIG. 3;

FIG. 5 is oblique view showing the underside of an upper bed and lower bed suitable for use with the bed platform shown in FIGS. 3 and 4;

FIG. 6 is a side view of a bed assembly incorporating the upper bed and lower bed of FIG. 5 disposed on the bed platform of FIGS. 3 and 4;

FIG. 7 shows a linear actuator assembly suitable for use with the present disclosure;

FIG. 8A is an oblique view of the underside of the lower bed and upper bed in a configuration wherein there is open space between the upper and lower beds;

FIG. 8B is an oblique view of the underside of the lower bed and upper bed in the same configuration from a different point of view;

FIG. 9A is an oblique view of the underside of the lower bed and upper bed, wherein the upper and lower beds are pulled together;

FIG. 9B is a side view of the bed assembly of FIG. 6, in the configuration shown in FIG. 9A;

FIG. 10A is an oblique view showing upper bed and lower bed in a third configuration wherein the space between the upper and lower beds are disposed at a maximum distance from one another;

FIG. 10B is a side view of the bed assembly of FIG. 6, in the configuration shown in FIG. 10A;

FIG. 11A is an oblique view of a body harness suitable for use with the spinal decompression device disclosed herein;

FIG. 11B is a second oblique view of a body harness suitable for use with the spinal decompression device disclosed herein;

FIG. 12 is a side view of a spinal decompression system, showing a patient disposed on the treatment bed;

FIG. 13A is a treatment profile graph showing certain treatment parameters over time;

FIG. 13B is a treatment profile graph showing certain treatment parameters over time;

FIG. 14A is an oblique view of the underside of the upper bed and lower bed, showing a pneumatic regulator system (PRS);

FIG. 14B is a side view of the treatment bed assembly, showing certain components of the pneumatic regulator system;

FIG. 15A is an oblique view of a lordotic support bladder;

FIG. 15B is an oblique view of an upper mattress;

FIG. 15C is an oblique view of an upper mattress having a lordotic support bladder disposed therein;

FIG. 16A is a front view of a spinal decompression control remote;

FIG. 16B is an oblique view of the spinal decompression control remote of FIG. 16A;

FIG. 17 is a front view of the spinal decompression control remote showing a graphic user interface (GUI) disposed thereon;

FIG. 18A is a side view of a treatment bed;

FIG. 18B is a side view of the treatment bed of FIG. 18A showing an inflated lordotic support bladder disposed therein;

FIG. 19A shows an upper oblique view of a pneumatic body harness and rigging;

FIG. 19B shows a lower oblique view of the pneumatic body harness and rigging of FIG. 6A;

FIG. 20A shows a top view of the pneumatic body harness shown in FIGS. 6A and 6B, in an uninflated state;

FIG. 20B shows a top view of the pneumatic body harness of FIGS. 6A-7A, in an inflated state;

FIG. 20C shows an upper oblique view of the pneumatic body harness shown in FIGS. 6A-7B, in an uninflated state; and

FIG. 20D shows an upper oblique view of the pneumatic body harness of FIGS. 6A-7C, in an inflated state.

### DETAILED DESCRIPTION OF THE DISCLOSURE

The following detailed description provides certain specific embodiments of the subject matter disclosed herein. Although each embodiment represents a single combination of elements, the subject matter disclosed herein should be understood to include sub-combinations of the disclosed elements. Thus, if one embodiment comprises elements A, B, and C, and a second embodiment comprises elements B and D, then the inventive subject matter is also intended to include other remaining combinations of A, B, C, or D, even if not explicitly disclosed herein.

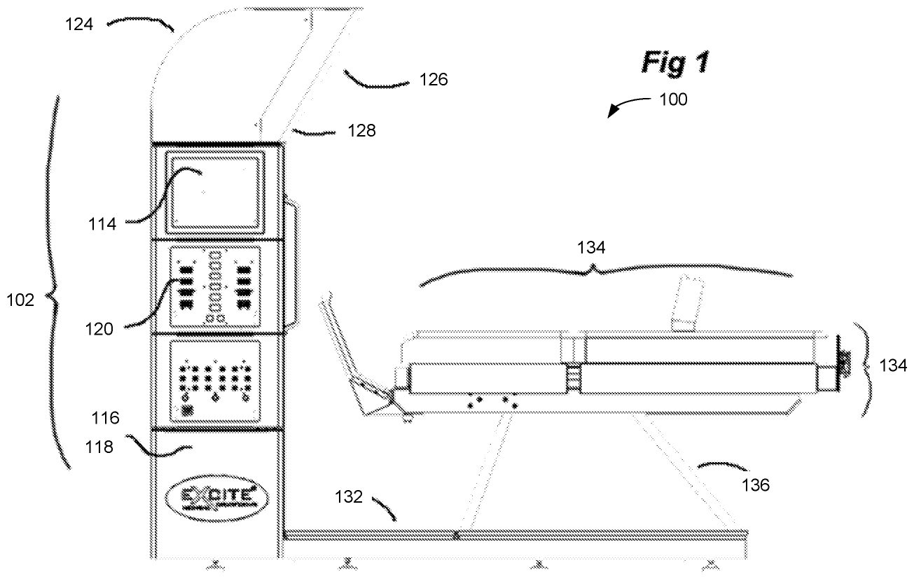

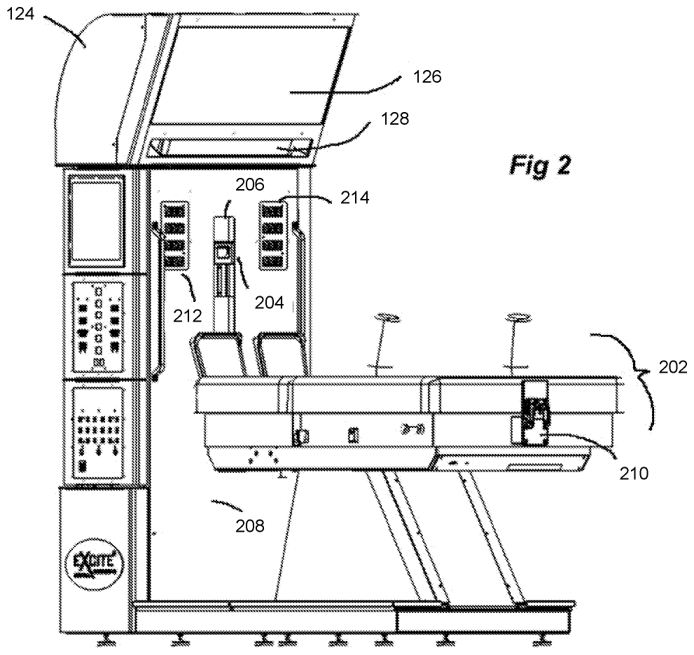

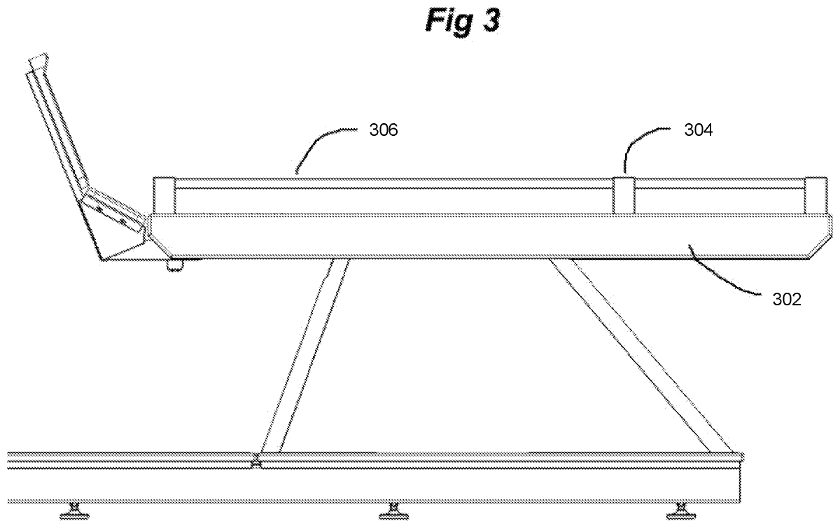

FIG. 1 is a side view of a spinal decompression system 100. FIG. 2 shows the spinal decompression system 100 of FIG. 3 from a front/end point of view. In one embodiment, spinal decompression system 100 is the DRX9000® manufactured by Excite Medical of Tampa Bay LLC in Tampa, Florida in the United States of America. In another embodiment, spinal decompression system 100 is the DRX9000-SL™, also manufactured by Excite Medical®.

Spinal decompression system 100 incorporates treatment tower 102. In one embodiment, treatment tower 102 incorporates a touch screen computer 114. Touch screen computer 114 incorporates a computer and touch screen monitor into a single chassis. This type of device is sometimes referred to as a “PanelPC.” Touch screen computer 114 incorporates a microprocessor and a memory-storage device containing custom software. Touch screen computer 114 uses the microprocessor, memory-storage device and custom software to control external devices, including the touch screen monitor.

As noted, touch screen computer 114 incorporates a custom touch screen monitor, allowing a healthcare provider to communicate with touch screen computer 114. This is accomplished through graphically-displayed and audibly-emitted information and by physical-data entry by the healthcare provider via the touch screen monitor. In one embodiment, touch screen computer 114 is an Advantech Computer with 15.6″ touch screen monitor, part no. TPC-1551WP-3S51.

In one embodiment of the present disclosure, treatment tower 102 contains servo-amplifier 116 and servomotor 118. In this embodiment, servo-amplifier 116 and servomotor 118 are attached to and disposed within the base of tower 102. Depending on the application, touch screen computer 114 may control servo-amplifier 116 through analog or digital, wired or wireless communications. Servo-amplifier 116 is in communication with servomotor 118 through servo-cables which provide power and communications between servo-amplifier 116 and servomotor 118. In one embodiment, servo-amplifier 116 is a Kollmorgen part number AKD-x00306 and servomotor 118 is a Kollmorgen model number AKM44E.

Touch screen computer 114 controls display-panel 120, which contains visual indicators reflecting information, including treatment data. In the embodiment shown in FIG. 1, visual indicators include 7-segment LED display modules which communicate treatment data to the healthcare provider through alphanumeric visual information. In one embodiment, the 7-segment LED display modules used are Kingbright 7-Segment Yellow LED Displays, part no. SA23-11YWA.

Touch screen computer 114 controls a Patient Engagement Device, utilized as an interactive biofeedback system 124 in one embodiment, shown mounted atop tower 102. Biofeedback system 124, in one embodiment of the present disclosure is a metal shroud formed to hold multi-media display 126 and speaker system 128. Biofeedback system 124 communicates treatment data and healthcare provider data through graphically-displayed and audibly-emitted information provided by the touch screen computer 114. One embodiment utilizes an LG 32″ LED Monitor part no. 32ML600M-B, and a BOSE SOUNDBAR no. 838309-1100 for this purpose.

Treatment tower 102 contains a power supply in communication with externally-supplied AC power. In certain embodiments, the power supply is a DC power supply. Typical AC power includes 90V to 264V 50/60 Hz, but may include voltages over 400V AC. Typical DC power supplies include those supplying one or more of 5 VDC, 12 VDC and 24 VDC, though other common voltages include the range extending from 1 VDC to above 48 VDC. In one embodiment, the DC power supply used in treatment tower 102 is a Mean Well 24 VDC 150 W, part number LRS-350-24.

Treatment tower 102 is mounted on base frame 132. Treatment bed 134 is mounted on base frame 132 adjacent to treatment tower 102, via pedestal 136. Pedestal 136 is secured in place to the base frame 132, as is treatment tower 102. Treatment bed 134 is positioned and fixed horizontally upon the top of pedestal 136 as shown. In a separate embodiment, treatment bed 134 may be mounted on an axle or similar mechanism which allows treatment bed 134 to tilt upwards to accommodate patients from a standing position.

Biofeedback system 124, multi-media display 126 and speaker system 128 are shown oriented to face a patient laying supine with their head positioned at head-end 202 of treatment bed 134. With or without a head-supporting pillow, the patient may naturally look downwards toward biofeedback system 124 as one might do watching evening-time television programs in their own beds.

As seen in FIG. 2, belt-lift pulley 204 is shown generally centered within vertically-oriented rectangular cutout 206 in front-facing panel 208. Belt-lift pulley 204 allows a tension strap to travel by rolling from within treatment tower 102 to a lower-body harness. In one embodiment, belt-lift holder assembly 204 moves vertically with respect to treatment tower 102 and behind front-facing panel 208 proximal to the patient and is actuated by a motor (not shown). Retention strap tightener 210 is shown mounted to the most distal facing end of the upper bed relative to treatment tower 102. Two separate display panels 212 and 214 are shown as integrated into front-facing panel 208. Display panels 212 and 214 are in communication with touch screen computer 114 and display information. In certain embodiments, alphanumeric information is displayed through an array of 7-segment display modules.

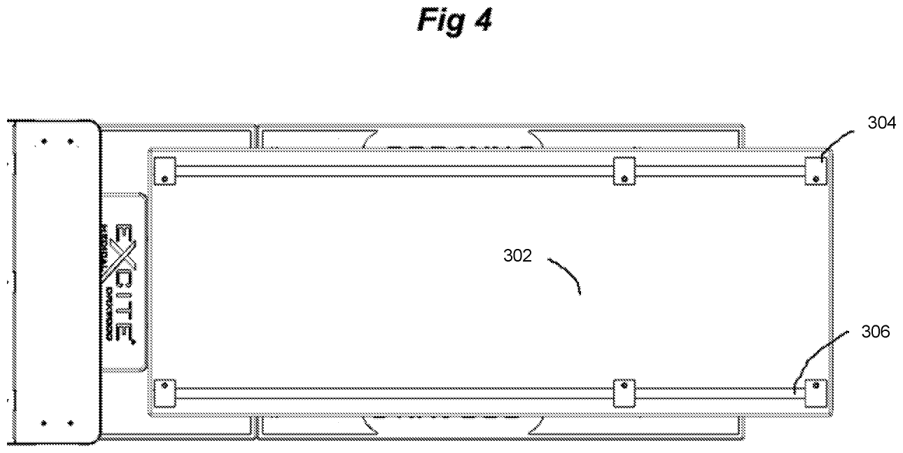

FIG. 3 is a side view of bed platform 302. FIG. 4 is a top view of bed platform 302 revealing both rows of support blocks 304 and rails 306 secured to bed platform 302. Bed platform 302 is one component of treatment bed 134. The construction of bed platform 302 can vary by application. In certain embodiments, bed platform 302 is a custom-formed and machined metal frame. A material found to be useful for this purpose is 10 gauge steel. The material may be stainless steel and may be powder-coated for additional protection and aesthetic appearance.

An array of support blocks 304 are secured to bed platform 302. In one embodiment, each row of support blocks 304 includes three support blocks 304. Support blocks 304 are secured to the top of bed platform 302, in-line along the length of the sides of bed platform 302, adjacent to the edge thereof. Support blocks 304 may be secured to bed platform 302 using threaded fasteners, rivets, adhesive, welding or other securement mechanisms known to those of skill in the art. Certain embodiments may use alternate methods of securement. A second row of support blocks 304 is positioned opposite the first row of support blocks 304 and similarly secured.

Rails 306 run through the support blocks 304 along either side of bed platform 302 and above the surface thereof. In the embodiment shown in FIG. 3, rails 306 are secured approximately 3 inches above the surface of bed platform 302. In the embodiment shown in FIG. 3, rails 306 are made of stainless steel. Rails 306 are secured in place by a suitable method of securement. As an example, rails 306 may be secured by threaded fasteners such as bolts, washers, nuts in certain embodiments of the present disclosure, but may be secured by alternate methods in other embodiments.

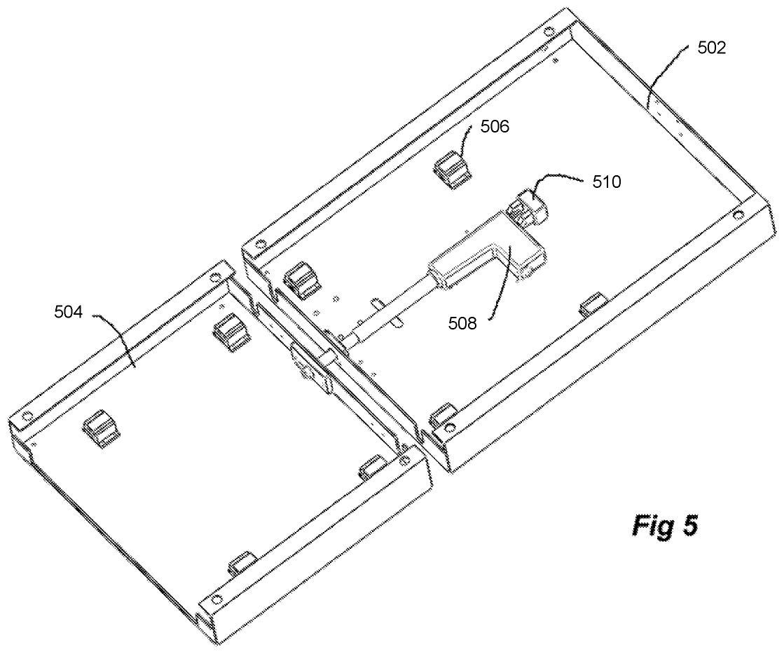

FIG. 5 shows the undersides of upper bed 502 and lower bed 504 arranged according to one embodiment of the teachings of the present disclosure. Together, upper bed 502, lower bed 504 and bed platform 302 form a treatment bed 134. Bearings disposed within pillow blocks 504 capture rails 306 supported by support blocks 304 affixed to the bed platform 302. This configuration allows both the upper bed 502 and lower bed 504 to move along rails 306.

In one embodiment, upper bed 502 is a custom-formed and machined metal frame, formed of 5052-H32 aluminum sheet metal 0.1875 inches thick. Upper bed 502 may be protectively and decoratively powder-coated. Upper bed 502 is sized to accommodate a patient's upper torso from approximately T11 to the top of the patient's head in overall length. In the embodiment shown, upper bed 502 incorporates a set of aluminum pillow blocks 506, each containing a bearing. Pillow blocks 506 are securely attached to the underside of upper bed 502, arranged in-line to each other along the length of each of the undersides of upper bed 502 symmetrically.

The width between the two rows of pillow blocks 506 their placement underneath upper bed 502 relative to its lengthwise centerline are matched to the width and positioning of rails 306 affixed to bed platform 302 via support blocks 304. Pillow blocks 506 are captured by and ride upon stationary rails 306. This configuration allows upper bed 502 to translate lengthwise over bed platform 302. The range of movement for upper bed 502 spans from a position approximately halfway along the bed platform 302, where it is closest to treatment tower 102, to a position at or beyond the distal edge of bed platform 302, where it is most distant from treatment tower 102.

Spinal decompression unit 100 may incorporate a motor to adjust the vertical position of upper bed 502 relative to bed platform 302 for patients' ease and comfort. Spinal decompression unit 100 is also operable to lock the position of upper bed 502 in place upon the rails 306 relative to bed platform 302 once the desired position is obtained.

Lower bed 504 has a similar construction to upper bed 502, described above. Lower bed 504 is designed to accommodate a patient's body beginning approximately about their sacrum and extending to and beyond their extended legs and feet. A knee support may be placed under a patient's knees, with its base laying upon the upper surface of lower bed 504, upon a cushion or mattress, or other most outward upper surface of the lower bed 504. This arrangement may be more comfortable for the patient and aids in proper lumbar spinal alignment during spinal decompression treatment.

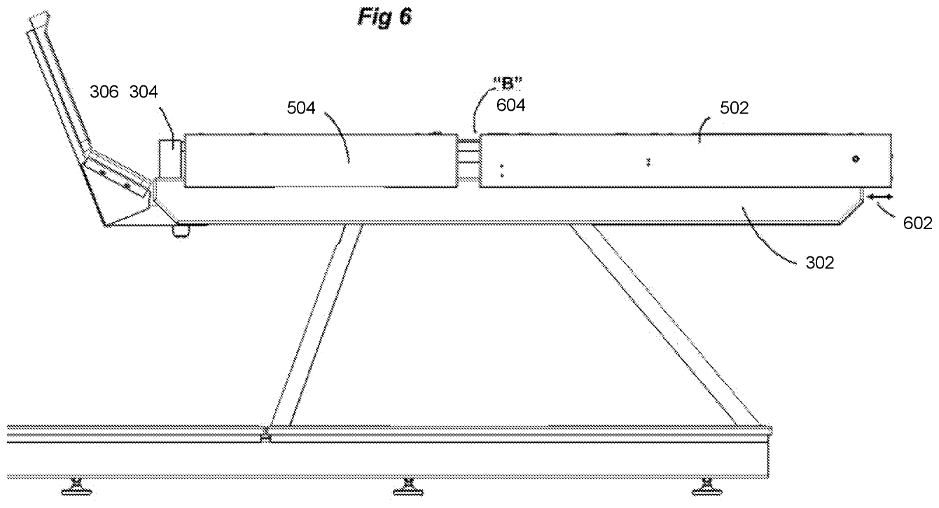

According to one embodiment of the present disclosure, lower bed 504 can move upon rails 306 and support blocks 304 bound by the upper bed 502 at its most distal position “B” 604 to, at, or beyond the most proximal edge of the bed-platform 302, as shown in FIG. 6. In certain embodiments, such movement may occur with minimal resistance. In this configuration, lower bed 504 may be said to ‘float’ or be ‘floating.’

With upper bed 502 locked into place relative to bed platform 302, lower bed 504 is otherwise allowed to float freely within its limits of travel. Motor 508, located in upper bed 502, is used to adjust the lower bound of the range of float of lower bed 504. In one embodiment, motor 508 is a linear motor, such as model LA31 manufactured by LINAK. In one preferred embodiment of the present disclosure, motor 508 is controlled by the treatment touchscreen computer 114 within treatment tower 102. In an alternate embodiment, motor 508 may be controlled by a wired and/or wireless handheld remote by the healthcare provider or other user. In other embodiments, motor 508 may be controlled by touchscreen computer 114 in combination with a handheld remote.

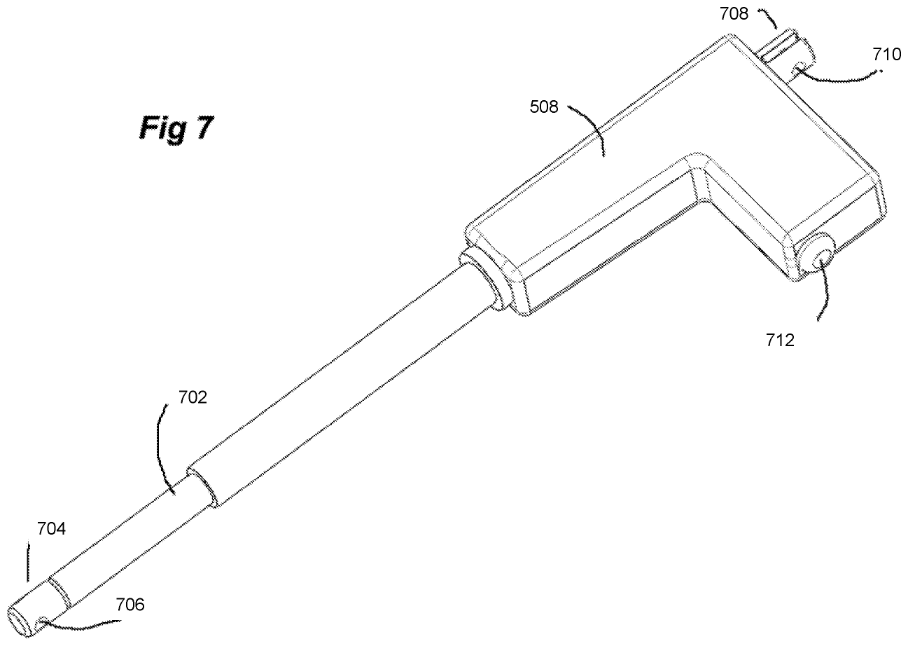

FIG. 7 is an oblique view of motor 508 and related hardware. Motor 508 is secured at one end to the underside of upper bed 502 via base post 708 which extends lengthwise from there. In one embodiment, base post 708 extends approximately 1.5 inches out from motor 508. Base post 708 may rotate at its connection point along the axis of extension and retraction of motor 508. A hole 710 through base post 708 allows for attachment to motor clevis 510 secured to the underside of upper bed 502. Motor clevis 510 may be manufactured from metal, such as 6061-T6 aluminum. Motor clevis 510 allows for attachment to base post 708. A similar clevis may be attached to linear motor rod end 704 via a pin or bolt through aperture 706.

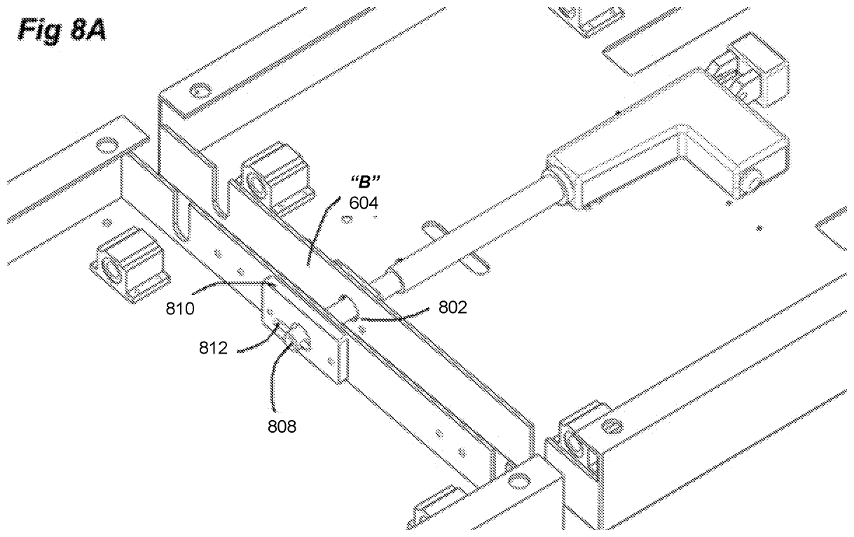

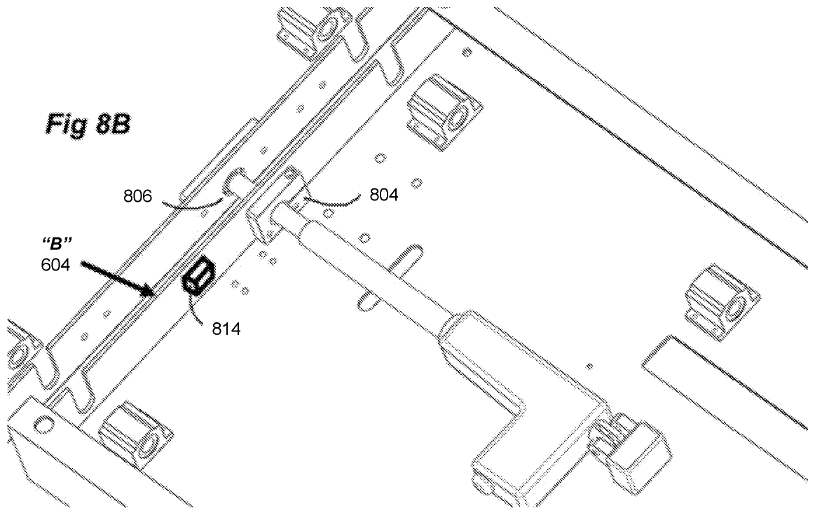

FIGS. 8A and 8B disclose additional detail as to the interface between upper bed 502 and lower bed 504. The end panel 604 (“B”) of upper bed 502 facing treatment tower 102 has a circular cutout 802 disposed therein, approximately at the center thereof. Cutout 802 provides and opening to allows a portion of motor 508 to extend therethrough. In the embodiment shown in FIGS. 8A and 8B, the main body of motor 508 is disposed within the envelope of upper bed 502, and linear motor rod 702 extends into lower bed 504.

Motor guide block 804 is secured to the inside face of the end panel “B” 604 of upper bed 502. Motor guide block 804 is disposed over circular cutout 802. A hole passing through motor guide block 804 allows either the body of linear motor 508 or linear motor rod 702 to pass therethrough with minimal clearance, thus aligning the orientation of motor 508 while allowing for axial movement through motor guide block 804. Motor guide block 804 may be made of metal, such as 6061-T6 aluminum, and may incorporate bushings or bearings to allow the linear extension and retraction of linear motor rod 702 while supporting the motor rod 702 radially. In an alternate embodiment, motor guide block 804 may be constructed from a polymer, and preferably a self-lubricating polymer with good machinability such as acetal, which is sold commercially by DuPont® under the brand name Delrin®.

Linear motor rod 702 passes through circular cutout 802 in upper bed 502 and through second circular cutout 806 in lower bed 504. Distal end 808 of linear motor rod end 704 passes through bed brake 810 and is secured in place by retention pin 812. Bed brake 810 may be constructed from a variety of materials, but rubber having a hardness in the range of 90A shore is known to be one suitable material for bed brake 810. A variety of retention pins 812 known to those skilled in the art are used to secure the end of the linear motor rod 704 as described above. Steel is an appropriate material for retention pin 812, and in particular, 316-series stainless steel.

Bed brake 810 is an approximately rectangular block having a hole passing from the lengthwise vertical face of one side completely through the opposite side approximately near the center of the block. The sizing of bed brake 810 is such that it is captured between the inside vertical face of lower bed 504 and retention pin 812 disposed in hole 706 in linear motor rod end 704.

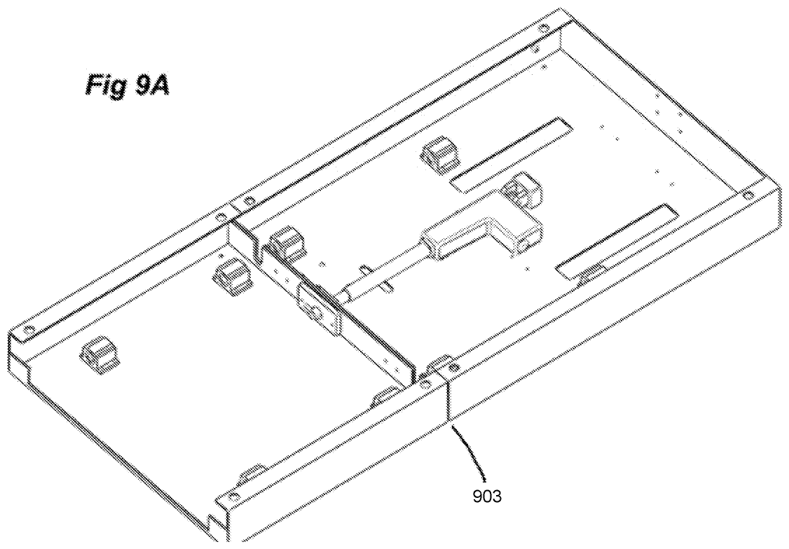

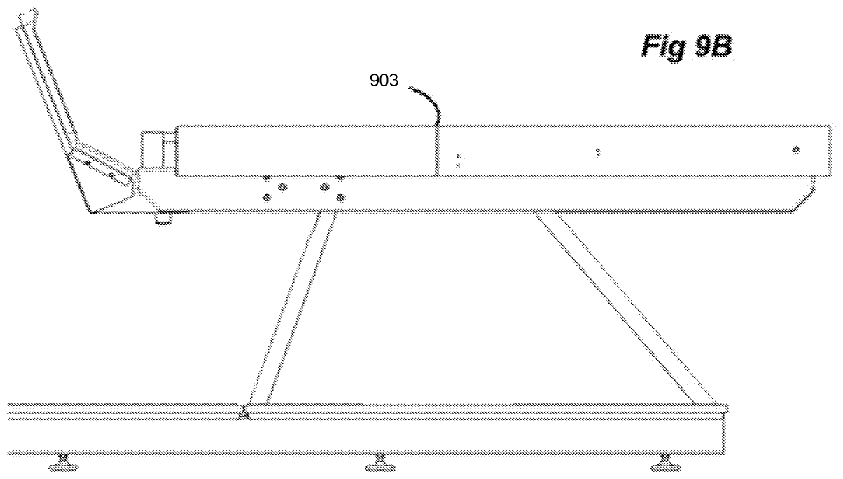

In operation, retraction of linear motor rod 702 pulls bed brake 810 closer to end panel 604 of upper bed 502. Further retraction of linear motor rod end 704 will pull lower bed 504 towards end panel 604 until the ends of lower bed 504 and upper bed 502 meet. The point of full contraction of motor 508, wherein there is no gap between the distal end of lower bed 504 and end panel 604 of upper bed 502 is referred to as a “locked” configuration. This configuration is shown in FIGS. 9A and 9B, showing the joint 903 between upper bed 502 and lower bed 504 in the locked configuration. When lower bed 504 is not in the locked position, it is referred to as being in an “unlocked” configuration.

Extension of linear motor rod end 704 of motor 508 allows bed brake 810 to travel lengthwise within lower bed 504 frame to the maximum extension of linear motor rod end 704. FIGS. 10A and 10B show one embodiment of the present disclosure in an “unlocked” configuration. At a maximum extension of motor rod 702, and with floating lower bed 504 positioned to the furthest extent allowed by the bed brake 810, an extension gap or space 1003, also identified as “X,” is formed between end panel 604 of upper bed 502 and the adjacent vertical surface of the lower bed 504. This extension space may be referred to herein as “X 1003,” “X-space 1003,” “extension-space 1003,” “X-space” or “X-SPACE™”.

In a preferred embodiment of the present disclosure, bed brake 810 only limits the maximum extension of lower bed 504 as it floats on rails 306 and support blocks 304 of the bed platform 302. A healthcare provider may instruct the touchscreen computer 114 to limit the maximum extension of bed brake 810 according to the specific needs of the spinal decompression patient at the time of treatment. The touchscreen computer 114 instructs motor 508 to extend and retract accordingly both before and during spinal decompression treatment.

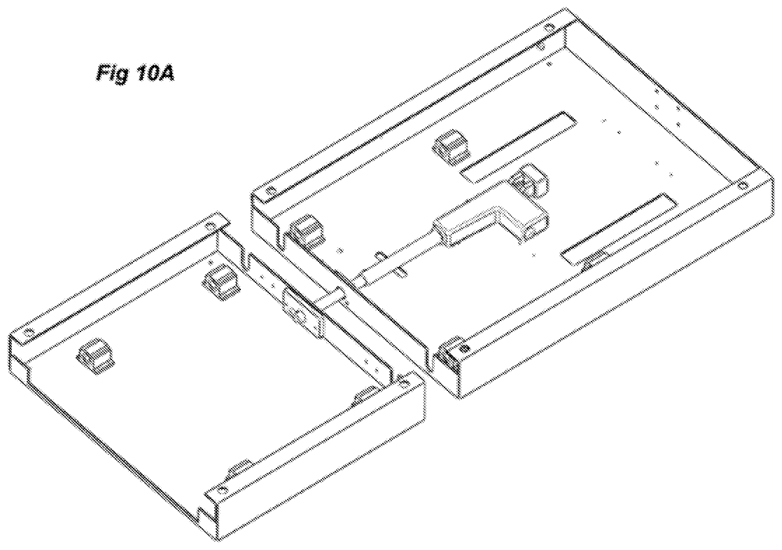

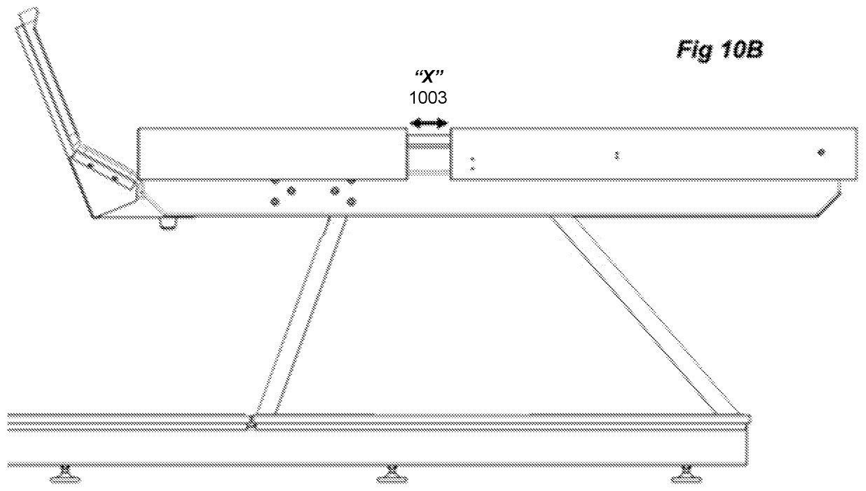

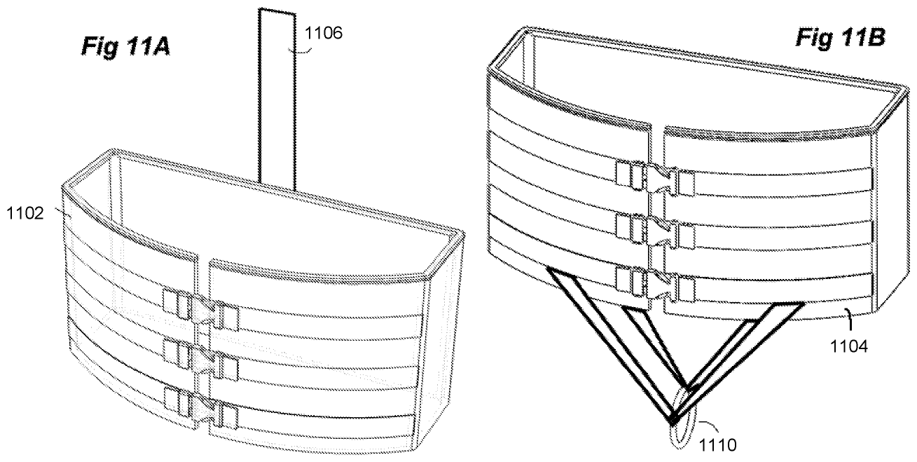

FIGS. 11A and 11B show two harnesses suitable for use with the teachings of the present disclosure. Patients are typically fitted with an upper-body harness 1102 made of cloth and other materials affixed to each other with stiches and adhesives. Upper-body harness 1102 contains straps which are used to cinch the harness to conform snuggly against the patient torso. Once a patient is positioned supine upon the treatment bed 134, with their head disposed away from treatment tower 102 on the upper bed 502, retention strap 1106 of upper-body harness 1102 is secured to the retention strap tightener 210 and fixed tight. In so doing, the patient's upper body, extending from approximately T10 to the top of the patient's head, is fixedly secured to the upper end of treatment bed 134.

At the time of fitting upper-body harness 1102, a separate lower-body harness 1104 is also fitted to the patient. Lower-body harness 1104 is similarly made of cloth and other materials affixed to each other with stitches and adhesives. Lower-body harness 1104 incorporates straps which are used to cinch lower-body harness 1104 to conform snuggly against the patient's lower torso in the waist region, hips region or sacral region.

In one embodiment, a set of straps is affixed on the front and rear, left and right sides of lower-body harness 1104, extending down to a stainless-steel decompression ring 1110, which may be secured there via stitching, adhesives, melting or other means known to those skilled in the art of textile design and manufacture. Decompression ring 1110 is centered between the patients' legs. A stainless-steel snap-clip is fastened to decompression ring 1110. The stainless-steel snap-clip is used to secure the decompression ring 1110 to the D-ring of the tension strap of treatment tower 102.

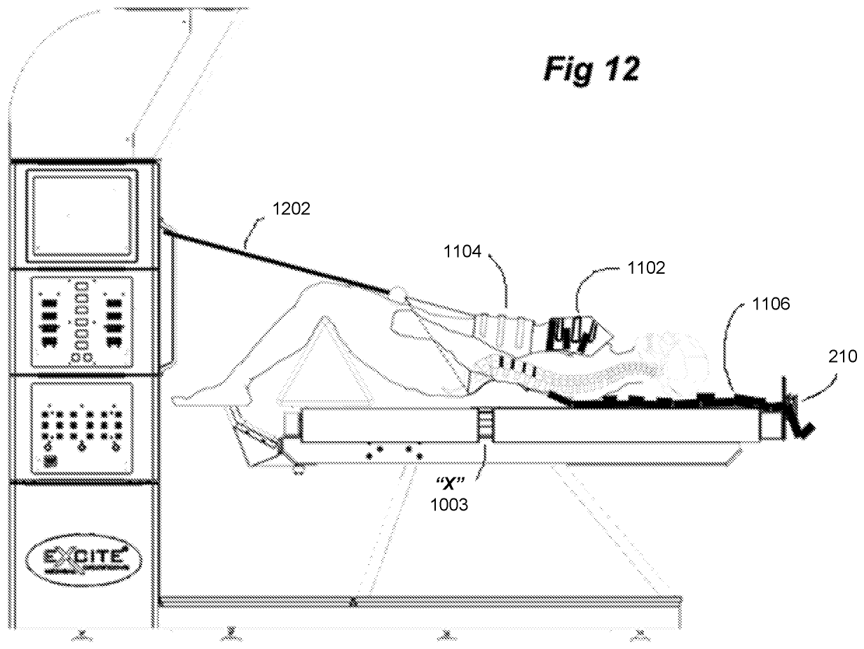

FIG. 12 depicts spinal decompression system 100 with a patient placed on treatment bed 134 for spinal decompression treatment. Within treatment tower 102, servo-amplifier 116 and servomotor 118 are utilized to rotate a decompression spool upon which is wound tension strap 1202, which may also be referred to as a ‘treatment strap’. In one embodiment, the width of tension strap 1202 varies between 0.5 inch and 6 inches wide. In particular embodiments, tension strap 1202 may be 2 inches or 4 inches wide. Tension strap 1202 is typically made of an organic, inorganic, or synthetic blending of materials that function approximately as a flexible, strong strap of appropriate thickness. Tension strap 1202 runs from the decompression spool, up over belt-lift pulley 204, exiting treatment tower 102 through rectangular cutout 206. Tension strap 1202 terminates in a secure attachment to an appropriate connection point, such as a stainless-steel D-ring. In one embodiment, belt-lift pulley 204 is actuated by a motor to move vertically with respect to treatment tower 102 behind front-facing panel 208.

A healthcare provider or touchscreen computer 114 may actuate motor 508 to move lower bed 504 into the locked position prior to placing the patient supine upon the treatment bed 134. Once the patient is positioned supine upon upper bed 502 and lower bed 504 of treatment bed 134, their upper body harness retention strap 1106 is secured to retention strap tightener 210.

The connection point of tension strap 1202 is typically used to clip a stainless-steel snap-clip extending from a decompression ring 1110 connected by straps to portions of lower-body harness 1104 described above. A healthcare provider may extend tension strap 1202 by pulling the connection point towards the patient in preparation for a spinal decompression treatment session. By pulling the connection point, a healthcare provider extends the tension strap 1202 towards the decompression ring 1110 of lower-body harness 1104. The connection point and decompression ring 1110 are then clipped securely to one another.

In certain applications, spinal decompression treatment may be initiated while lower bed 504 remains in the locked position prior to the unlocking of lower bed 504. In other applications, lower bed 504 may be unlocked and allowed to float according to instructions from a healthcare provider or touchscreen computer 114. The spacing between upper bed 502 and lower bed 504 may be set according to a pre-determined patient-specific spacing prior to the initiation of spinal decompression treatment. In this situation, the lower bed 504 may be left to float for a period of time and at a spacing 1003 which is set before treatment. Depending on the details of the treatment required, these parameters can vary programmatically or as desired by a healthcare provider throughout the duration of a spinal decompression treatment.

X-space 1003 between upper bed 502 and lower bed 504 will typically range between zero inches and 4 inches. In certain, embodiments X-space 1003 may extend to more than 6 inches. X-space 1003 is the limit of spinal elongation that the lumbar spine may elongate during a spinal decompression cycle at maximum tension. For example, if the maximum X-space 1003 at any given point is limited to 2 inches by the healthcare provider or touchscreen computer 114 then that is the most that the patient's spine can elongate at that time.

The compliance of the patient's spine during the cycling of tension by servomotor 118 is directly affected by the maximum length to which the patient's spine is allowed to elongate at any given time. The allowable X-space 1003, controlled using the disclosure described herein, could be made by a healthcare professional, the result of a calculation by touchscreen computer 114, or determined by the patient themselves.

Setting the X-space 1003 for a patient's spine can become particularly important when the patient's spine is heavily guarded by its paraspinal muscles. This situation is commonly encountered in patients experiencing chronic lumbar back pain. The present disclosure provides a process by which the patient's spine can be encouraged to lengthen and relax by only a small amount initially. As the patient returns for successive treatment sessions at a later time, the healthcare provider or touchscreen computer 114 may elect to extend the X-space 1003 if they observe the spine is healing sufficiently to progress to more therapeutic levels of intervertebral disc elongation.

In another embodiment the present disclosure describes a process of varying the X-space 1003 progressively during the spinal decompression treatment. For example, a typical modern spinal decompression treatment profile may consist of several cycles of maximum and minimum tension applied to tension strap 1202. In one embodiment, the healthcare provider or preferably the touchscreen computer 114 may unlock lower bed 504 only slightly during the first or initial tension cycles. This initial phase may be followed by increases in the X-space 1003 of lower bed 504 on successive treatment cycles. This approach allows the patient's spine to accommodate at its own rate to spinal elongation and relaxation cycles, and also limits the X-space 1003 in the initial phases as a safety precaution.

Linear motor rod 702 may be extended and retracted to adjust X-space 1003 in essentially real-time. X-space 1003 may be set according to inputs from touchscreen computer 114, healthcare provider or the patient, depending on the application. In one embodiment, an optical distance sensor module 814, such as an infrared proximity sensor, may be secured to upper bed 502 behind end panel B 604, as shown in FIG. 8B. Distance sensor 814 is oriented and positioned such that it can sense the proximity of lower bed 504 through a cutout in end panel B 604. This allows distance sensor 814 to interact with the corresponding end surface of lower bed 504 immediately opposite X-space 1003. In one embodiment, the optical distance sensor 814 is an Infrared Proximity Distance Sensor manufactured by Sharp, part number GP2Y0A51SK0F 2-15CM.

The outputs from the optical distance sensor module 814 reflecting the proximity of upper bed 502 to lower bed 504 are communicated to touchscreen computer 114. This data may be displayed on touchscreen monitor 112, on 7-segment display modules 122 to the healthcare provider and to the patient via biofeedback system 124.

The healthcare provider may at any time adjust the degree of X-space 1003 from its maximum extension down to zero using the touchscreen-monitor 112 of the Touchscreen computer 114. Touchscreen computer 114 may be instructed to unlock 1003 the lower bed 504 by adjusting the motor 508 in the upper bed 502 and using the outputs from optical distance sensor module 814.

As noted above, the degree of X-space 1003 may be progressively increased or decreased during the course of a single spinal decompression treatment. The degree to X-space 1003 may be either increased or decreased in response to patient feedback during the course of treatment. Biofeedback system 124 may be used to provide alphanumeric information graphically to the user to advise the patient in advance as to adjustments in the direction and intensity of tension they can expect and the amount of lumbar spinal elongation they may experience as a function of the progressive increase in X-space 1003 or other treatment parameters.

The patient may be provided with a mechanism for controlling motor 508 in upper bed 502. The patient may be provided with some degree of control over X-space 1003 adjustment and may use that control in concert with information gathered through the biofeedback system 124 to manage their own individual rate of treatment progression. With patient training, a patient-controlled X-space 1003 feature in combination with a biofeedback system 124 may be deployed to locations where a trained healthcare provider may not be available, but a patient-specific spinal decompression experience is advantageous.

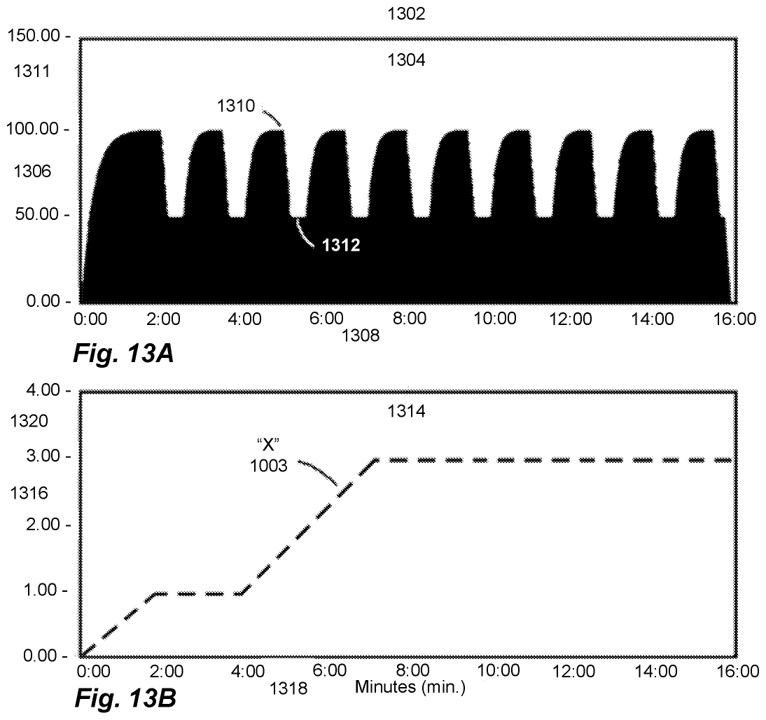

FIGS. 13A and 13B are graphs showing the values of certain spinal decompression parameters over time. FIG. 13A shows a treatment profile graph 1302 containing a spinal decompression intensity profile 1304. Treatment profile graph 1302 may be displayed on touchscreen computer 114 and on the biofeedback system 124 and may include audible voice-prompts. Along left vertical y-axis 1306, the intensity of the tension applied to the tension strap 1202 is shown in pounds, ranging from zero at the bottom to one-hundred and fifty pounds at the top of y-axis 1311. In certain embodiments, servomotor 118 can safely maintain tension levels up to and above 300 pounds. Along the bottom of graph 1302, x-axis 1308 shows time in minutes.

After the initiation of treatment, servomotor 118 is powered to rotate the decompression spool in a clockwise and counterclockwise direction, so as to alternate the direction and intensity of the tension applied to the tension strap 1202 in a series of maximum tension plateaus 1310 and minimum tension plateaus 1312. The maximum tension plateaus 1310 cause the patient's lumbar spine and specifically its lumbar-intervertebral discs, to actually elongate relative to their thoracic spine, and its thoracic-intervertebral discs, as the upper body is stationary and the lower body and thus the lower bed 504 are floating upon rails 306 and support blocks 304 affixed the bed platform 302. The degree of spinal elongation experienced during the maximum tension plateaus 1310 and minimum tension plateaus 1312 is constrained by X-space 1003, which is controlled by the degree of extension of linear motor rod 702 at any given time. Within the range of free motion available to lower bed 504, lower bed 504 moves in concert with the actual lumbar spinal elongation and proportionally to the direction and intensity of the tension applied to the tension strap 1202.

FIG. 13B shows a graph 1314 illustrating another treatment profile, showing the progression of X-space 1003 over the course of treatment. On the left vertical y-axis 1316, the length of the X-Space 1003 is shown in inches ranging from zero to 4 inches 1320. In alternate embodiments, X-space 1003 may include an upper limit of 6 inches. X-axis 1318 shows time in minutes. Graph 1314 of the maximum allowable spinal elongation 1003 details a slow stepped progression from zero inches to a 3 inch maximum X-space 1003 over several maximum tension cycles 1310 and minimum tension cycles 1312.

Other forms of information may be graphically displayed on spinal decompression device 100. As an example, a graph could be displayed showing the actual real-time outputs from optical distance sensor 814. Treatment data may be captured and stored digitally. Such data may include the treatment profile graph 1302 and the patient settings. The actual outputs of optical distance sensor 814 may also be analyzed to suggest modifications to future patient spinal decompression treatment profiles 1302 and to automatically populate fields in the custom software 110 upon initiation of the next patient treatment session.

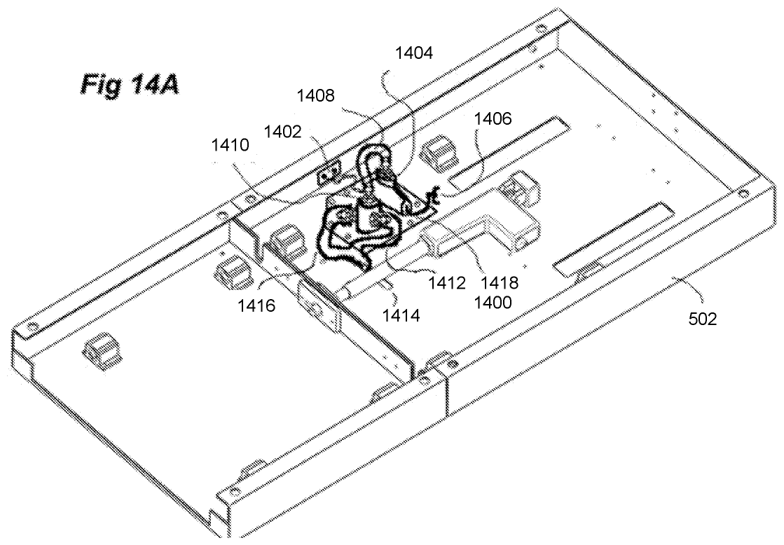

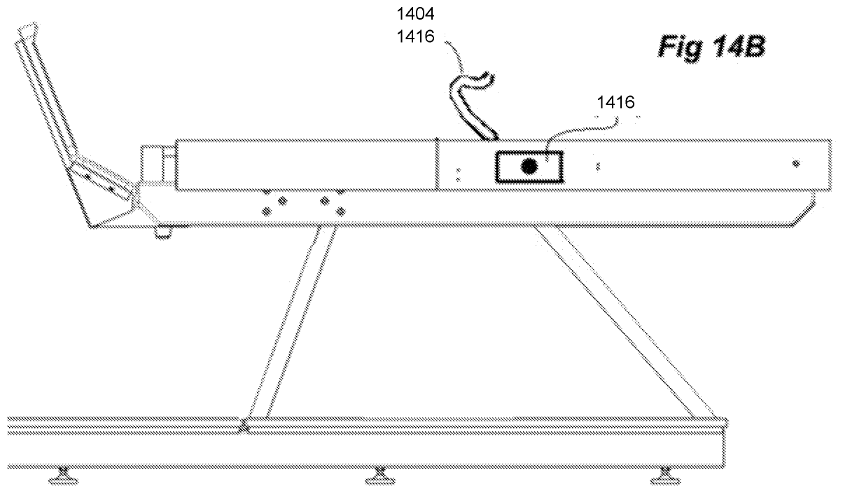

FIG. 14A shows the underside of an additional embodiment of upper bed 502, incorporating pneumatic regulator system (PRS) 1400. FIG. 14B shows upper bed 502 from a side view. Pneumatic regulator system (PRS) 1400 uses an air pump 1404 to pressurize or depressurize inflation hoses 1412 and 1416 to inflate and deflate one or more components of spinal decompression device 100. As an example, PRS 1400 may be used to inflate and deflate an inflatable lordotic support bladder. PRS 1400 is assembled onto PRS armature 1418, which is attached to the underside of the upper panel of upper bed 502. In one embodiment, PRS armature 1418 is secured using stainless steel hardware.

In one embodiment, air pump 1404 receives electric power through wired port 1406. Port 1406 may be in communication with and/or be controlled by touch screen computer 114. Air pump 1404 may contain one or more internal digital pressure sensors. Alternately, one or more digital pressure sensors may be incorporated elsewhere in the assembly. Digital pressure sensor data may be utilized to recall a patient's previous settings or used in a feedback loop for maintaining a certain fill pressure of a lordotic support bladder.

In certain embodiments, air pump 1404 may receive instructions via panel-mounted momentary push-button switch 1402. In this configuration, when the healthcare provider holds switch 1402 down, air pump 1404 is turned ‘on’. Pressurized air from air pump 1404 is forced through pressure hose 1408 and into air manifold 1410 connected by tubing ports, which allow two-way airflow. In one embodiment, the tubing ports may be NPT glass-filled nylon barbed ports.

In certain embodiments, manifold 1410 is a pneumatic short-circuit with one positive and one negative-pressure triggered check valve. When air pump 1404 is driven in a forward-biased manner to pressurize manifold 1410, a positive-pressure-triggered one-way check-valve opens at y-tube hose 1412, allowing positive pressure to build. A negative-pressure-triggered one-way check-valve closes at y-tube hose 1416, which forces air to pass through to the topside of upper bed 502 through inflation hose 1412.

When air pump 1404 is driven in a reverse-biased manner to depressurize manifold 1410, a negative-pressure-triggered one-way check-valve opens at y-tube hose 1416 side, allowing negative pressure to be generated. A positive-pressure-triggered one-way check-valve closes at y-tube hose 1412 side, which forces air to pass through from the topside of upper bed 502 through deflator-hose 1416, back through pressure-hose 1408, and exhausted to ambient by air pump 1404.

In certain embodiments, air pump 1404 may receive instruction and/or power from an external handheld control remote, which may be connected to air pump 1404 via panel mount connector 1402. In some embodiments, the patient may be provided with a mechanism for controlling the pressurization state of a lordotic support bladder real-time and during treatment, with either full control or with a limited degree of adjustment. Treatment data, including the air pressure and other patient settings, may be captured digitally and be used to automatically populate fields in the custom software 110 upon initiation of the next treatment session for that patient.

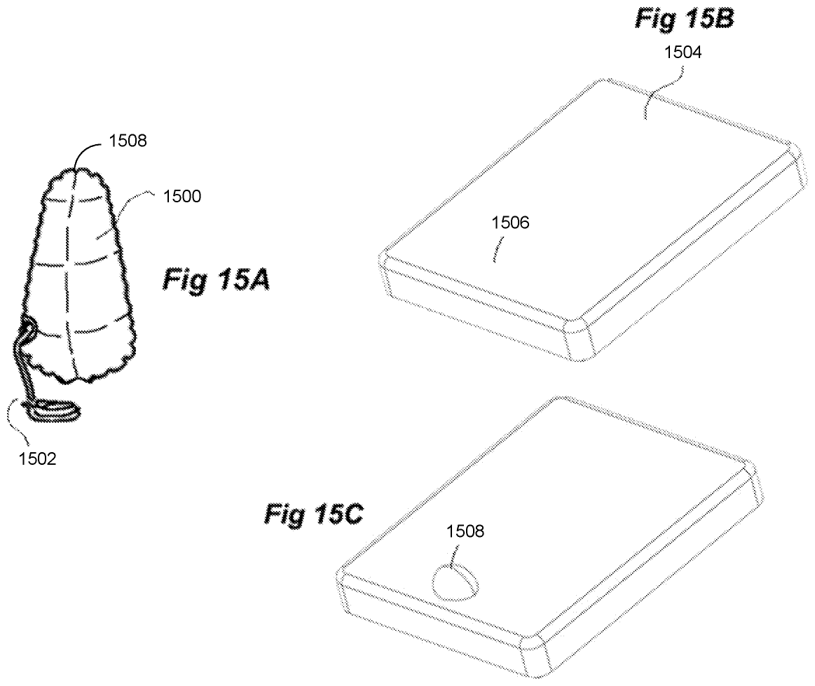

FIG. 15A is an oblique view of inflatable lordotic support 1500 according to one embodiment of the present disclosure. Inflatable lordotic support 1500 is shaped preferentially to conform to an apex of lordosis located at the peak 1508 of inflatable lordotic support 1500. A flexible air tube or hose 1502 is connected to the base of inflatable lordotic support 1500. Hose 1502, in turn, connects to inflator hose 1412 and deflator hose 1416.

FIGS. 15B and 15C are oblique views of upper mattress 1504 in two different states. FIG. 15B shows upper mattress 1504 incorporating inflatable lordotic support 1500 at position 1506. In this view, lordotic support 1500 is deflated. Thus, the upper surface of upper mattress 1504 is flat and generally planar with no protrusion.

FIG. 15C is an oblique view of upper mattress 1504 as shown in FIG. 15B after inflatable lordotic support 1500 is pressurized and inflated, resulting in the formation of a supporting feature configured to have a peak 1508 located preferentially under a patient's apex of lordosis.

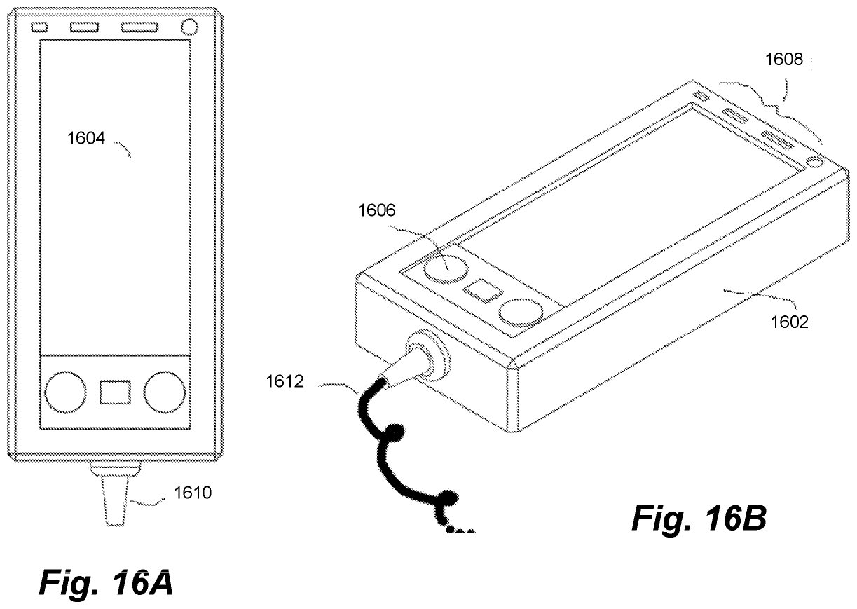

FIGS. 16A and 16B illustrate one embodiment of a handheld remote control 1600 according to the present disclosure. Handheld remote control 1600 is disposed within housing 1602. Housing 1602 allows for integration of touchscreen display 1604. Buttons 1606 at the base of handheld remote control 1600 provide additional methods of interaction for users. Upper panel 1608 at the top of handheld remote control 1600 may include a variety of components, including a speaker, microphone, flashlight and indicator lamp, as examples.

Handheld remote control 1600 may communicate via a wireless antenna 1610, or a wired connection 1612. Wired connection 1612 which may also provide electrical power to handheld remote control 1600. In certain embodiments, wireless hand controller 1600 may house batteries for powering of itself and external components.

In certain embodiments, handheld remote control 1600 may incorporate a microprocessor, digital memory and custom software to control various components of spinal decompression device 100. Such components may include motor 508 and air pump 1404, as examples. Handheld remote control 1600 may communicate audio and visual information to a user via display 1604. Handheld remote control 1600 may also communicate information via one or more indicator lamps and possibly a speaker disposed in upper panel 1608. Handheld remote control 1600 may receive input from a user through touchscreen 1604, buttons 1606 or a microphone in upper panel 1608. In the embodiment shown, button 1606A may be used to fully open lower bed 504 and button 1606B may be used to close and fully lock lower bed 504.

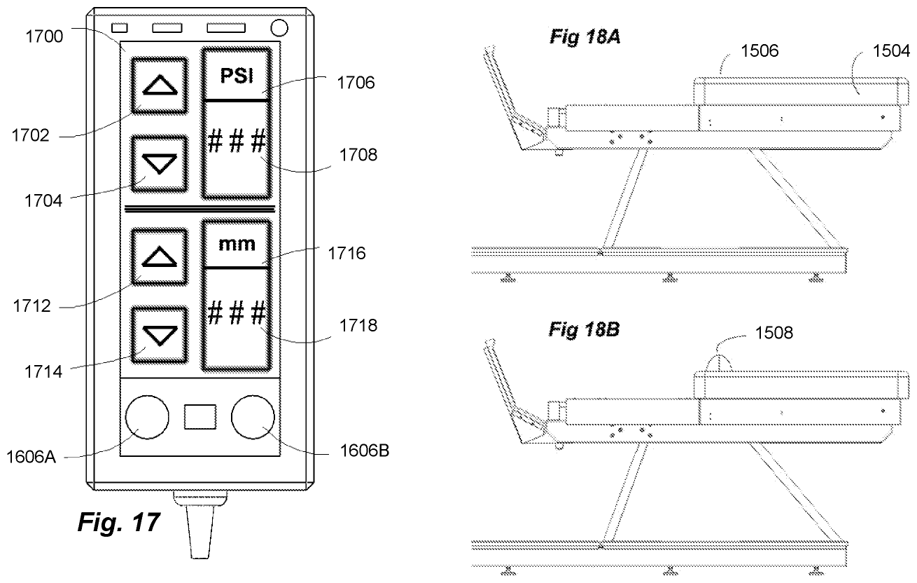

FIG. 17 shows a graphical user interface (GUI) 1700 displayed on touchscreen display 1604 of handheld remote control 1600. At the top of display 1604, up arrow 1702 and down arrow 1704 are shown to the left of PSI field 1706. PSI field 1706 indicates the degree of pressurization of the inflatable lordotic support 1500 as monitored by a pressure sensor. Numeric display field 1708 is disposed below PSI display field 1706. Numeric display field 1708 is operable to display a 3-digit numerical measurement reflecting the air pressure of inflatable lordotic support 1500.

At the bottom of display 1604, up arrow 1712 and down arrow 1714 are shown to the left of X-Space display field 1716. X-Space display field 1716 identifies the width of “X-Space” 1003 of the lower bed 504 as monitored by distance sensor 814. Numerical display field 1718 is disposed below X-Space display field 1716. Numerical display field 1718 is operable to display a 3-digit numerical measurement reflecting the width of X-space 1003.

FIGS. 18A and 18B are side views of treatment bed 134, showing upper mattress 1504 in two different states. FIG. 18A shows upper mattress 1504 when inflatable lordotic support bladder 1500 is uninflated. FIG. 18B shows upper mattress 1504 when inflatable lordotic support bladder 1500 is inflated. The peak 1508 of the surface of upper mattress 1504 is preferably aligned to a properly positioned patient's apex of lordosis.

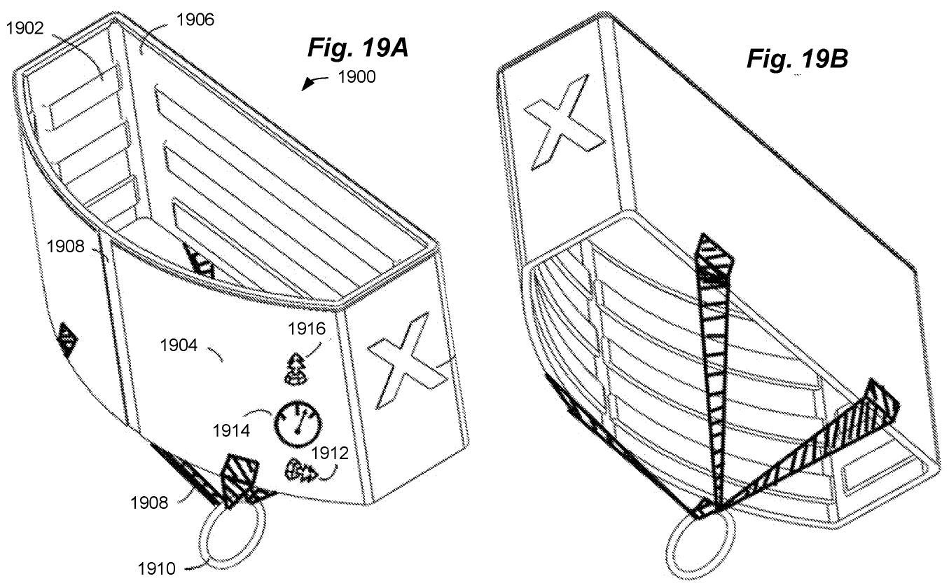

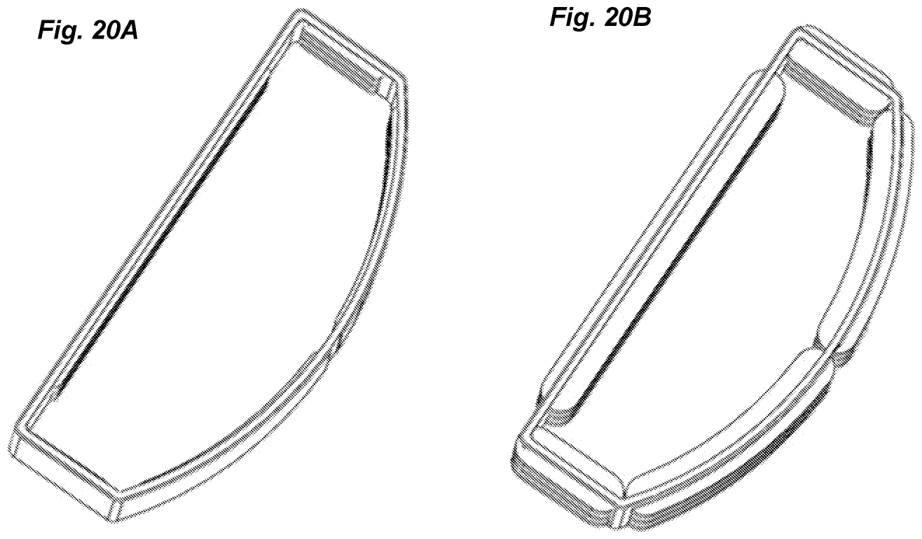

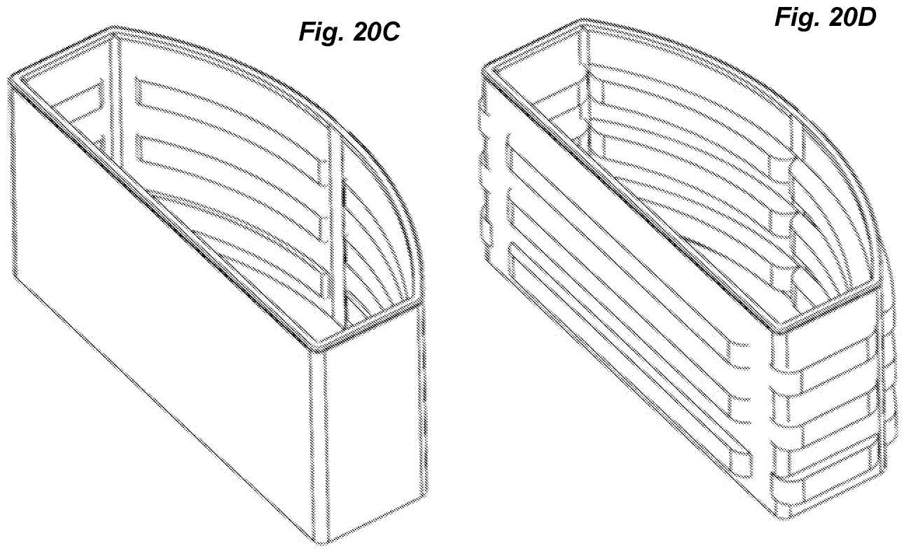

FIGS. 19A, 19B, 20A, 20B, 20C and 20D show various views of a pneumatic body harness 1900 and its functions, and also show the deflated and inflated states of body harness 1900. Pneumatic body harness 1900 may be used in place of upper body harness 1102, lower body harness 1104, or both, depending on the application. Pneumatic body harness 1900 is made of cloth and cloth-like materials, which may include cotton, nylon, Kevlar, and canvas. Other materials may include rubber, silicon rubber, foams, gels, plastic, metal, and Velcro to make other structures including pocket liners, rigid supports, pull-cords and handles, grips etc. The materials may be joined to each other with stiches and adhesives, sonic welding, vacuum forming, sealing, melting and other methods known to those skilled in the art.

Pneumatic body harness 1900 functions by increasing and decreasing how firmly a portion of a patient's body, including the waist region, hips region or sacral region, is secured by varying the air pressure inside of inflatable structures within its inner spaces. One embodiment of pneumatic body harness 1900 incorporates a network of inflatable pocket spaces 1902 sewn or formed into the inner space along the circumference of body harness 1900. In one other embodiment a stiffer, less elastic outer material 1904, such as ballistic nylon, may be used to form the outside of body harness 1900 while more conforming and elastic material forms inward facing material 1906. With this construction, as inflatable pocket spaces 1902 are inflated, their increase in volume is forced inwards towards the patient's body. In other embodiments, inflatable inner tubes, such as are used in bicycle tires, may be sewn or formed into the inner space of the pneumatic body harness 1900. As the innertubes are inflated, the space between them is reduced, which results in a firmer grip about the patient's body.

Pneumatic body harness 1900 reduces or eliminates the need for straps and buckles as a securing mechanism. Optimally, pneumatic body harness 1900 reduces or eliminates the need for a break in continuity of treatment. Patients may find that putting on a lower-body harness is complicated by a multitude of straps and buckles to be secured and adjusted. In other scenarios, a patient may not possess sufficient mobility to successfully pull the harness up from their feet. In this case, a gap can be provided in the harness to allow the harness to be worn like a belt or cummerbund. While a gap or split can be incorporated into pneumatic body harness 1900, with said gap being closed using straps and buckles, the adjustment and tightening of pneumatic body harness 1900 would be achieved through the use of air pressure within segmented inflatable structures in the innerspaces of body harness 1900.

Pneumatic body harness 1900 may incorporate internal check valve inlet 1912 for an attachment means, such as a nozzle, to supply pressurized air. Internal check valve inlet 1912 may prevent unintended depressurization of body harness 1900. A separate pressure-relief check-valve 1916 may be placed on the upper-facing side 1904 of the body harness 1900 so that when a patient is positioned supine upon treatment bed 134, check valve 1916 is clear to release any over-pressurization harmlessly upwards to the ambient environment. Pneumatic body harness 1900 also incorporates pressure gauge 1914 to ensure consistent pressurization between treatments for each patient. Pressure gauge 1914 also serves to indicate if pneumatic body harness 1900 is capable of being pressurized or if it has malfunctioned.

In certain embodiments, pneumatic body harness 1900 is available in a variety of starting internal diameters which are capable of evenly reducing the inner diameter up to a final diameter via inflatable structures 1902 affixed to and within body harness 1900. In this way, each ‘size’ of body harness 1900 can be specified as having a starting and a final internal diameter. In another embodiment, the disclosure herein is specified to customers as having an initial and a final waist size as is customary with conventional clothing. One may extend this principle to include other inflatable structures 1902 within body harness 1900 which act to manipulate a patient's spine.

Pneumatic body harness 1900 is suitable to endure routine sustained exposure to industry-standard spinal decompression tension levels including up to 150 lbs. In other embodiments, maximum tension levels may include up to 500 lbs. of tension. Body harness 1900 utilizes a similar set of four straps 1908 affixed to its front and rear, left and right sides, which extend down to a decompression ring 1910 and is secured there via stitching, adhesives, melting and other means known to those skilled in the art of textile design and manufacture. In this arrangement, body harness 1900 is suitable for use as a pelvic harness. By rearranging the connections, body harness 1900 could alternately be employed as an upper-body harness.

Pneumatic body harness 1900 is designed to be secured around a patient with minimal inflation, if any. It may be provided with sufficient inflation to remain in place around the patient's torso without external support. A user may inflate pneumatic body harness 1900 using an appropriately configured external air or pressure source while standing if necessary. By design, pneumatic body harness 1900 is meant to be inflated to levels sufficient for use during treatment only after the patient is positioned supine on treatment bed 134.

FIGS. 20A, 20B, 20C and 20D show a comparison between deflated and inflated states of pneumatic body harness 1900 as relates to changes in overall internal diameter. Straps 1908 and decompression ring 1910 are not shown in these figures. In certain embodiments, the maximum fill pressure may be less than 60 pounds per square inch. In other embodiments, the maximum fill pressure is up to and may exceed 120 psi. In additional embodiments, the maximum fill pressure may be less than 30 psi.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refers to at least one of something selected from the group consisting of A, B, C . . . and N, the text should be interpreted as requiring only one element from the group, not A plus N, or B plus N, etc.

1. A spinal decompression device comprising:
a treatment bed having a first end, a second end and a mattress;
a treatment tower disposed at the first end of the treatment bed; and
an inflatable lordotic support bladder disposed within the mattress of the treatment bed.

2. The spinal decompression device of claim 1 wherein a pressure within the inflatable lordotic support bladder may be dynamically adjusted during treatment.

3. The spinal decompression device of claim 1 wherein the device can store and recall treatment settings specific to a patient.

4. The spinal decompression device of claim 1 wherein the device applies a treatment based on treatment parameters, and wherein the treatment parameters may be adjusted based on biofeedback.

5. The spinal decompression device of claim 1 wherein the device applies spinal decompression tension to the spinal column of a patient, and wherein the tension varies over time.

6. The spinal decompression device of claim 1 wherein the inflatable lordotic support is aligned to a location corresponding to an apex of lordosis of a patient.

7. The spinal decompression device of claim 1 wherein a pressure within the inflatable lordotic support may be adjusted manually during treatment.

8. A spinal decompression device comprising:
a treatment bed having a first end, a second end and a mattress;
a treatment tower disposed at the first end of the treatment bed;
an inflatable lordotic support bladder disposed within the mattress of the treatment bed; and
an air pump, disposed within the treatment bed, operable to inflate the inflatable lordotic support bladder.

9. The spinal decompression device of claim 8 wherein a pressure within the inflatable lordotic support bladder may be dynamically adjusted during treatment.

10. The spinal decompression device of claim 8 wherein the device can store and recall treatment settings specific to a patient.

11. The spinal decompression device of claim 8 wherein the device applies a treatment based on treatment parameters, and wherein the treatment parameters may be adjusted based on biofeedback.

12. The spinal decompression device of claim 8 wherein the device applies spinal decompression tension to the spinal column of a patient, and wherein the tension varies over time.

13. The spinal decompression device of claim 8 wherein the inflatable lordotic support is aligned to a location corresponding to an apex of lordosis of a patient.

14. The spinal decompression device of claim 8 wherein a pressure within the inflatable lordotic support may be adjusted manually during treatment.

15. A spinal decompression device comprising:
a treatment bed having a first end, a second end, an upper mattress and a lower mattress;
a treatment tower, having a tension strap, disposed at the first end of the treatment bed;
an inflatable lordotic support bladder disposed within the upper mattress of the treatment bed; and
an air pump, disposed under the upper mattress, operable to dynamically inflate and deflate the inflatable lordotic support bladder.

16. The spinal decompression device of claim 15 wherein a pressure within the inflatable lordotic support bladder may be dynamically adjusted during treatment.

17. The spinal decompression device of claim 15 wherein the device can store and recall treatment settings specific to a patient.

18. The spinal decompression device of claim 15 wherein the device applies a treatment based on treatment parameters, and wherein the treatment parameters may be adjusted based on biofeedback.

19. The spinal decompression device of claim 15 wherein the device applies spinal decompression tension to the spinal column of a patient via the tension strap, and wherein the tension applied varies overtime.

20. The spinal decompression device of claim 15 wherein the inflatable lordotic support is aligned to a location corresponding to an apex of lordosis of a patient.
